(19) United States
(12) Patent Application Publication
(10) Pub. No.: US 2006/0029573 A1
(43) Pub. Date: Feb. 9, 2006

(54) Pegylated interferon alpha-1b

(76) Inventors: Chun Shen, San Diego, CA (US); Ying Buechler, Carlsbad, CA (US); Xiaochun Chen, Carlsbad, CA (US); Dawn X. Wen, San Diego, CA (US); Yixin Wang, Shenzhen (CN); Shehui He, Shenzhen (CN); Qianlan Wang, Shenzhen (CN)

Correspondence Address: PAUL, HASTINGS, JANOFSKY & WALKER LLP, P.O. BOX 919092, SAN DIEGO, CA, 92191-9092, US

(21) Appl. No.: 11/172,409

(22) Filed: Jun. 30, 2005

Related U.S. Application Data
(60) Provisional application No. 60/584,504, filed on Jun. 30, 2004.
(60) Provisional application No. 60/689,155, filed on Jun. 9, 2005.

(51) Int. Cl. A61K 38/21; C07K 14/56

(52) U.S. Cl. 424/85.7; 530/351

(57) ABSTRACT

The invention provides PEG-IFN α-1b conjugates, where a PEG moiety is covalently bound to Cys⁸⁶ of human IFN α-1b conjugates. A pharmaceutical composition and a method for treating inflammatory diseases, infections, and cancer are also provided. The invention further relates to a method for the modification of interferons by conjugation of a PEG moiety to free cysteine residues in interferon molecules.

### RELATED APPLICATIONS

This application claims the benefit of priority of U.S. Provisional Application Ser. No. 60/584,504 filed Jun. 30, 2004 and U.S. Provisional Application Ser. No. 60/689,155 filed Jun. 9, 2005 the disclosures of which are incorporated herein by reference in their entirety for any purpose.

### FIELD OF THE INVENTION

The present invention relates generally to the modification of human interferon to increase serum half-life and a pharmacokinetic profile, in vivo biological activity, stability, and reduce immune reaction to the protein in vivo. More specifically, the invention relates to the site-specific covalent conjugation of monopolyethylene glycol to a free thiol group (Cys⁸⁶) of human interferon alpha-1b. The present invention also relates to processes for cysteine-specific modification of interferons and as well as their use in the therapy, treatment, prevention amelioration and/or diagnosis of bacterial infections, viral infections, autoimmune diseases and conditions, inflammatory processes and resultant diseases or conditions, and cancers.

### BACKGROUND OF THE INVENTION

Interferons

Interferons are a family of naturally occurring small proteins and glycoproteins produced and secreted by most nucleated cell, e.g. in response to viral infection and other antigenic stimuli. Interferons display a wide range of antiviral, antiproliferative, and immunomodulatory activities on a variety of cell types and have been used to treat many diseases including viral infections (e.g., hepatitis C, hepatitis B, HIV), inflammatory disorders and diseases (e.g., multiple sclerosis, arthritis, asthma, cystic fibrosis, interstitial lung disease) and cancer (e.g., myelomas, lymphomas, liver cancer, breast cancer, melanoma, hairy-cell leukemia) and have been also applied to other therapeutic areas. Interferons render cells resistant to viral infection and exhibit a wide variety of actions on cells. They exert their cellular activities by binding to specific membrane receptors on the cell surface. Once bound to the cell membrane, interferons initiate a complex sequence of intracellular events, including the induction of enzymes, suppression of cell proliferation, immunomodulating activities such as enhancement of the phagocytic activity of macrophages and augmentation of the specific cytotoxicity of lymphocytes for target cells, and inhibition of virus replication in virus-infected cells.

Interferons (IFNs) have been classified into at least four groups according to their chemical, immunological, and biological characteristics: alpha (leukocyte), beta (fibroblast), gamma, and omega. Interferons are known to affect a variety of cellular functions, including DNA replication and RNA and protein synthesis, in both normal and abnormal cells. Thus, cytotoxic effects of interferon are not restricted to tumor or virus infected cells but are also manifested in normal, healthy cells as well. As a result, undesirable side effects arise during interferon therapy, particularly when high doses are required. Administration of interferon can lead to myelosuppression resulting in reduced red blood cell, white blood cell and platelet levels. Higher doses of interferon commonly give rise to flu-like symptoms (e.g., fever, fatigue, headaches and chills), gastrointestinal disorders (e.g., anorexia, nausea and diarrhea), dizziness and coughing.

α Interferons

HuIFN-αs are encoded by a multigene family consisting of about 20 genes which encode proteins having approximately 80-85% of amino acid sequence homology. HuIFN-α polypeptides are produced by a number of human cell lines and human leukocyte cells after exposure to viruses or double-stranded RNA, or in transformed leukocyte cell lines (e.g., lymphoblastoid lines).

Beginning in 1986, the U.S. Food and Drug Administration (FDA) has approved a number of interferon drugs including INF α-2b and INF α-2a for the treatment of chronic hepatitis, chronic myeloid leukemia, and hairy cell leukemia.

Interferon α-1b The primary sequence of interferon α-1b was first published by Mantei et al. in 1980 (Gene 10: 1-10) incorporated herein by reference in their entirety) (GenBank Accession No. NM_{—}024013.1; GI: 13128949; and GenBank Accession No. NP_{—}076918.1; GI:13128950). Interferon α-1b has been identified as a 166-amino acid, single chain polypeptide, which shares 83% homology with interferon α-2a and interferon α-2b. Interferon α-1b comprises five cysteine residues at amino acid positions 1, 29, 86, 99, and 139. In its native conformation, interferon α-1b forms 2 pairs of intra-molecular disulfide bonds (between Cys¹-Cys⁹⁹; Cys²⁹-Cys¹³⁹), leaving a free thiol group at the Cys⁸⁶ residue (Weissmann et al, 1982, Structure and expression of human IFN-α genes, Phil. Trans. R. Soc. Lond. B. 299:7-28).

Interferon α-1b has been reported to have the same biological and therapeutic properties as interferons α-2a and α-2b including immunomodulating, anti-viral and anti-cancer properties. IFN α-1b has been tested in clinical trials with hundreds of patients in China to determine therapeutic properties and adverse reactions. Interferon α-1b (Sinogen) was the first recombinant protein drug to be approved in 1992 by the Ministry of Public Health of China. and Nagata et al., in 1980 (Nature 287:401-408) (the contents of which are Interferon α-1b (Sinogen) has been used for more than 10 years to treat several million patients with hepatitis B, hepatitis C, viral infections, and cancers.

PEGylation of Interferons

Interferons may be administered parenterally for various therapeutic indications. However, parenterally administered proteins may be immunogenic, and may have a short pharmacological half life. Consequently, it can be difficult to achieve therapeutically useful blood levels of the proteins in patients. These problems may be overcome by conjugating the proteins to polymers such as polyethylene glycol.

Covalent attachment of the inert, non-toxic, bio-degradable polymer polyethylene glycol (PEG), also known as polyethylene oxide (PEO), to molecules has important applications in biotechnology and medicine. PEGylation of biologically and pharmaceutically active proteins has been reported to improve pharmacokinetics resulting in sustained duration, improve safety (e.g., lower toxicity, immunogenicity and antigenicity), increase efficacy, decrease dosing frequency, improve drug solubility and stability, reduce proteolysis, and facilitate controlled drug release.

Therapeutic PEG-protein conjugates currently in use include: PEGylated adenosine deaminase (ADAGEN®, Enzon Pharmaceuticals) used to treat severe combined immunodeficiency disease; pegylated L-asparaginase (ONCAPSPAR®, Enzon Pharmaceuticals) used to treat acute lymphoblastic leukemia; and pegylated interferon α-2b (PEG-INTRON® Schering Plough) and pegylated interferon α-2a (PEGYSYS, Roche) used to treat hepatitis C. See Burnham, Am. J. Hosp. Pharm., 15:210-218 (1994) for a general review of PEG-protein conjugates with clinical efficacy (which is incorporated herein by reference in its entirety).

Attaching PEG to reactive groups found on the protein is typically done utilizing electrophilically-activated PEG derivatives. For example, PEG may be attached to the ε-amino groups on lysine residues and α-amine on the N-terminus of polypeptide chains.

Generally, PEG conjugates consist of a population containing a variable number of PEG molecules attached per protein molecule (“PEGmers”) ranging from zero to the number of amino groups in the protein, or containing one PEG molecule attached to a variable site per protein molecule (positional isomers). Non-specific PEGylation, however, can result in conjugates that are partially or virtually inactive. Reduction of activity may be caused by shielding the protein's active receptor binding domain. For example, PEGylation of recombinant IFN-β and IL-2 with a large excess of methoxy-polyethylene glycolyl N-succinimidyl gluterate and methoxy-polyethylene glycolyl N-succinimidyl succinate reportedly results in increased solubility, but also a reduced level of activity and yield.

Therapeutic pegylated interferon alphas (IFN α) are mixtures of positional isomers that have been mono-pegylated at specific sites on the core IFN α-2b molecules (Grace et al, 2001, J. Interferon and Cytokine Research 21:1103-1115) and on the core IFN α-2a (Bailon et al, 2001, Bioconjugate Chem 12:195-202; Monkarsh et al, 1997, Analytical Biochemistry 247:434-440). The in vitro anti-viral and anti-proliferative activity is varied resulting from the site of pegylation and size of PEG attached (Grace et al, 2005, J. Biological Chemistry, 280:6327-6336).

Site-Specific PEGylation.

α-amine of the N-terminal of a polypeptide is a single site to be pegylated depending upon whether the N-terminal is involved in the active receptor binding domain. For example, α-amine of the N-terminal of G-CSF is mono-pegylated, retaining biological activity (U.S. Pat. No. 5,824,784, Kinstler, O. B. et al, 1998, “N-terminal Chemically Modified Protein Compositions and Methods”). α-amine of the N-terminal Cys¹ of interferon α-2b is mono-pegylated, exhibiting the lowest biological activity in STAT translocation assay as compared to that of His³⁴, Lys¹³⁴, Lys⁸³, Lys¹³¹, Lys¹²¹, Lys³¹ to be monopegylated (Grace et al, 2005, J. Biological Chemistry, 280:6327-6336).

Site-specific mono-PEGylation of proteins is a desirable goal, yet most proteins do not possess a specific native site for the attachment of a single PEG polymer, other than α-amine of the N-terminal of a protein or a free cysteine residue of a protein. It is therefore likely that PEGylation of a protein will produce isomers that are partially or totally inactive.

Thiol-selective PEG derivatives have been reported for site-specific PEGylation. A stable thiol-protected PEG derivative in the form of a parapyridyl disulfide reactive group was shown to specifically conjugate to the free cysteine in the protein, papain. The newly formed disulfide bond between papain and PEG could be cleaved under mild reducing conditions to regenerate the native protein. PEG-IFN-β conjugates have been reported in which a PEG moiety was covalently bound to Cys¹⁷ of human IFN-β, by a process of site specific PEGylation with a thiol reactive PEGylating agent orthopyridyl disulfide (Patent WO 99/55377 (PCT/US99/09161), El Tayar, N., et al, 1999, “Polyol-IFN-Beta Conjugates”).

PEG IFN-α Conjugates

European Patent Application EP 593 868 (which is incorporated by reference herein in its entirety) describes the preparation of PEG-IFN-α conjugates. The PEGylation reaction described in this patent was not site-specific, and therefore a mixture of positional isomers of PEG-IFN-α conjugates were obtained (see also Monkarsh et al., ACS Symp. Ser., 680:207-216 (1997), which is incorporated herein by reference in its entirety).

There is, thus, a need for site specifically modified PEG IFN-α conjugates, particularly α-1b conjugates, and methods for their production, to supplement the arsenal of pharmaceutical interferons available for treating human disease.

The entire disclosures of the publications and references cited herein are incorporated by reference herein in their entirety and are not admitted to be prior art.

### SUMMARY OF THE INVENTION

The present invention provides polyol-interferon-α conjugates having a polyol moiety covalently bound to Cys⁸⁶ of human interferon α-1b. Interferon may be isolated from human cells or tissues, or may be a recombinant protein expressed in a host, such as a bacterial cell, a fungal cell, a plant cell, an animal cell, an insect cell, a yeast cell, or a transgenic animal.

According to the present invention, the polyol moiety can for example, be a polyethylene glycol moiety or polyalkylene glycol moiety. In certain embodiments, the polyol-interferon α-1b conjugate of the present invention has the same or higher in vivo interferon-α activity as native human interferon α-1b. The polyol-interferon α-1b conjugate will, in a preferred aspect of the invention, have no other positional isomers and a homogenous molecular weight.

The present invention also provides pharmaceutical compositions, comprising a polyol-interferon-α conjugate having a polyol moiety covalently bound to Cys⁸⁶ of human interferon α-1b, and a pharmaceutically acceptable carrier, excipient or auxiliary agent.

Methods for producing a polyol-interferon conjugates are also provided in which an interferon that has a single free cysteine is conjugated with a maleimide polyol or a maleimide bis-polyol to form a covalent bond between the polyol and the free cysteine.

The method can be used to produce conjugates of naturally occurring, genetically engineered (e.g., recombinant), site-specific mutated, and chimeric interferons, including conjugates of human alpha interferon, such as recombinant human interferon α-1 b.

Methods are also provided for modulating processes mediated by interferon-α and for treating patients with an interferon-α-responsive condition or disease, comprising administering to a patient an effective amount of a polyol-interferon α-1b. The processes, diseases and conditions may include: inflammation, viral infection, bacterial infection or cancer. More specifically, the processes, diseases and conditions may be hepatitis C infection, hepatitis B infection, HIV infection, multiple sclerosis, arthritis, asthma, cystic fibrosis, interstitial lung disease, myeloma, lymphoma, liver cancer, breast cancer, melanoma, and hairy-cell leukemia.

### BRIEF DESCRIPTION OF THE DRAWINGS

The following drawings are illustrative of embodiments of the invention and are not meant to limit the scope of the invention as encompassed by the claims.

FIGS. 1A, 1B and 1C show the nucleotide sequence (FIG. 1A), amino acid sequence (FIG. 1B) and alignment (FIG. 1C) of the nucleotide and amino acid sequences of a human interferon α-1b.

FIGS. 2A and 2B show the conjugation mechanisms for Cys⁸⁶-specific monopegylation of interferon α-1b with a single chain mPEG (20 kD)-maleimide (FIG. 2A) and a branched chain mPEG2 (40 kd)-maleimide (FIG. 2B). The double bond of a maleimide undergoes an alkylation reaction with a sulfhydryl group to form a stable thioether bond. One of the carbons adjacent to the maleimide double bond undergoes nucleophilic attack by the thiolate anion to generate the addition product. At pH 7, the reaction of the maleimide with sulfhydryls proceeds at a rate 1000 times greater than its reaction with amines.

FIG. 3 shows SDS-PAGE electrophoresis of mPEG-IFN α-1b conjugates. Lanes 1 and 5 show protein molecular weight markers; lane 2 shows an unmodified IFN α-1b; lane 3 shows a mPEG (20 kD)-IFN α-1b conjugate; and lane 4 shows a mPEG2 (40 kD)-IFN α-1b conjugate.

FIGS. 4A, 4B, and 4C show size exclusion HPLC profiles of: an unmodified IFN α-1b (FIG. 4A); mPEG (20 kD)-IFN α-1b conjugate (FIG. 4B); and a mPEG2 (40 kD)-IFN α-1b conjugate (FIG. 4C).

FIGS. 5A and 5B show matrix-assisted laser desorption ionization (MALDI) time-of-flight (TOF) mass spectra of a mPEG (20 kD)-IFN α-1b conjugate (FIG. 5A), and a mPEG2 (40 kD)-IFN α-1b (FIG. 5B).

FIGS. 6A, 6B, and 6C show cation exchange HPLC profiles of: an unmodified IFN α-1b (FIG. 6A); a mPEG (20 kD)-IFN α-1b conjugate (FIG. 6B); and a mPEG2 (40 kD)-IFN α-1b conjugate (FIG. 6C).

FIG. 7 shows a characterization scheme of the Cys⁸⁶-specific monopegylation of IFN α-1b. A purified mPEG (20 kD)-IFN α-1b conjugate was digested by endoproteinase Glu-C, generating a Cys⁸⁶-pegylated peptide. The Cys⁸⁶-pegylated peptide was isolated by reverse phase HPLC using a gradient of acetonitrile/TFA, and further purified by size-exclusion HPLC. The purity of Cys⁸⁶-pegylated peptide was analyzed by SDS-PAGE and reverse phase HPLC. The molecular weight of the Cys⁸⁶-pegylated peptide was determined by SDS-PAGE and MALDI-mass spectroscopy. The Cys⁸⁶-specific monopegylation of the peptide was confirmed by N-terminal sequencing.

FIGS. 8A and 8B show reverse phase HPLC profiles of endoproteinase Glu-C peptide mapping tracings of an unmodified IFN α-1b (FIG. 8A), and a mPEG (20 kD)-IFN α-1b (FIG. 8B). The 29.1 minute peak is indicated as an unmodified Cys⁸⁶-containing peptide (FIG. 8A), while the 43.7 minute peak is indicated as a Cys⁸⁶-pegylated peptide (FIG. 8B).

FIG. 9 shows a pharmacokinetic profile of unmodified IFN α-1b, mPEG (20 kD)-IFN α-1b and mPEG2 (40 kD)-IFN α-1b conjugates in rats following a single subcutaneous administration.

FIG. 10 shows in vivo anti-tumor activities of mPEG (20 kD)-IFN α-1b conjugate and unmodified IFN α-1b in athymic Balb/C nude mice subcutaneously implanted with human renal tumor ACHN cells. Insert shows the dosages of mPEG (20 kD)-IFN α-1b conjugate and unmodified IFN α-1b used in the treatment of the mice implanted with the tumor. X- and y-axes indicate the weeks and the corresponding tumor volume, respectively.

### DETAILED DESCRIPTION OF THE INVENTION

The present invention is based on the discovery that the attachment of a polyol moiety, specifically a PEG moiety, to the Cys⁸⁶ residue of human IFN α-1b preserves IFN α-1b biological activity of native human interferon α-1b. Thus, not only does IFN α-1b with a polyol moiety attached to the Cys⁸⁶ residue exhibit IFN α-1b biological activity but this polyol-IFN α-1b conjugate also can provide the desirable properties conferred by the polyol moiety, such as improved pharmacokinetics, and reduced antigenicity.

The free thiol group (Cys⁸⁶) of interferon α-1b is available for sulfhydryl-specific conjugation, e.g., to polyethylene glycol. In addition, conjugation via maleimide-thiol is highly specific in mild neutral aqueous solutions. Thiol-specific monopegylation avoids the heterogeneity of positional isomers, which results from pegylation of multiple sites, such as pegylation via lysine residues.

Unless specific definitions are provided, the nomenclature utilized in connection with, and the laboratory procedures, techniques and methods described herein are those known in the art to which they pertain. Standard chemical symbols and abbreviations are used interchangeably with the full names represented by such symbols. Thus, for example, the terms “carbon” and “C” are understood to have identical meaning. Standard techniques may be used for chemical syntheses, chemical analyses, pharmaceutical preparation, formulation, delivery, and treatment of patients. Standard techniques may be used for recombinant DNA methodology, oligonucleotide synthesis, tissue culture and the like. Reactions and purification techniques may be performed e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), Harlow & Lane, Antibodies: A Laboratory Manual (Cold Spring Harbor Laboratory Press, Cold Spring Harbor, N.Y. (1988)), which are incorporated herein by reference in their entirety for any purpose.

“IFN-α” or “Interferon-α”, as used herein, means human leukocyte interferon, as obtained by isolation from biological fluids, cells, tissues, cell cultures or as obtained by recombinant DNA techniques in prokaryotic or eukaryotic host cells, including but not limited to bacterial, fungal, yeast, mammalian cell, transgenic animal, transgenic plant and insect cells, as well as salts, functional derivatives, precursors and active fractions thereof.

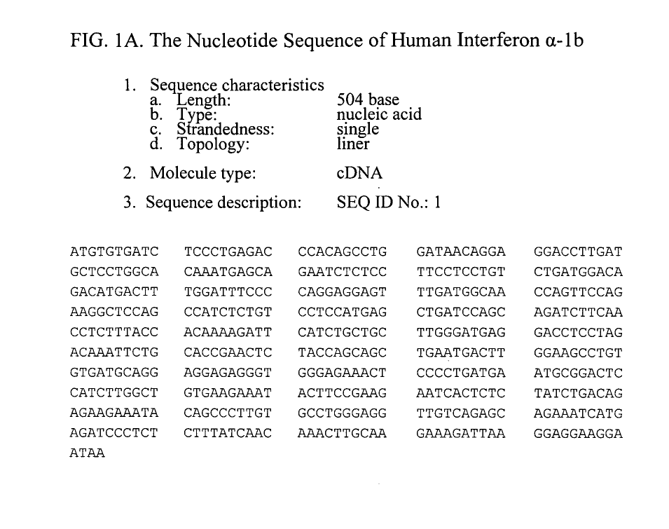

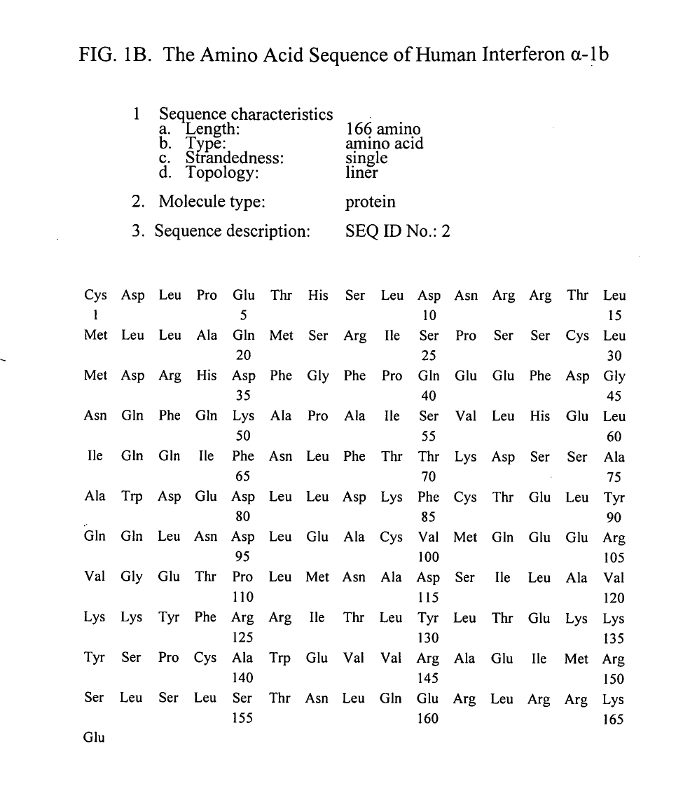

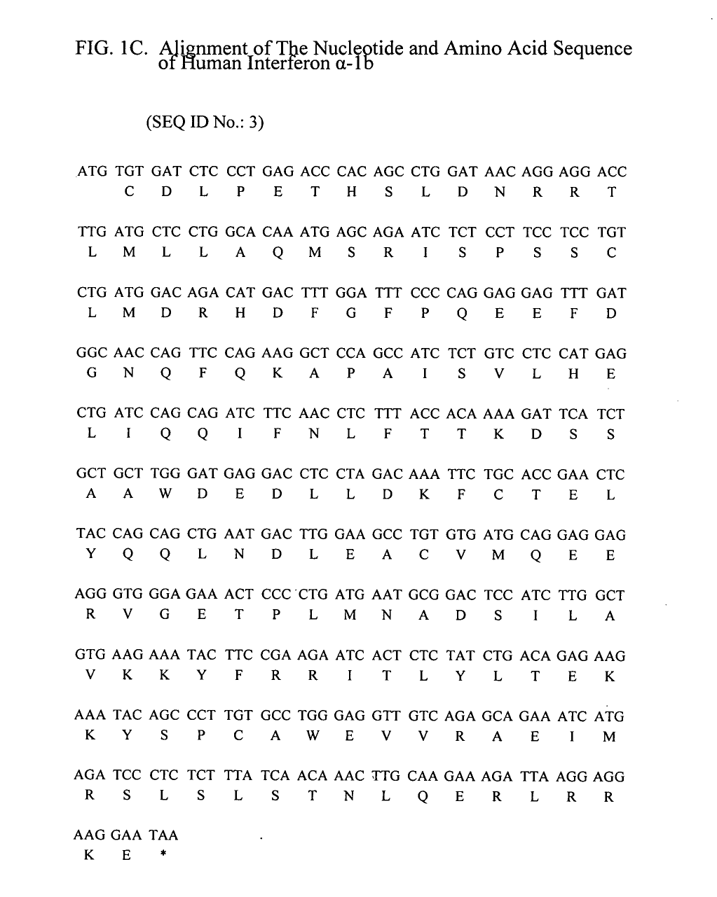

“Human IFN α-1b” refers to proteins having the amino acid sequence given as SEQ ID NO.:2 (FIG. 1B) or identified in GenBank Accession No.: NP_{—}076918.1 GI:13128950. The nucleotide sequence for a human IFN α-1b is shown in FIG. 1A (SEQ ID NO.1) and identified in GenBank Accession No.: NM_{—}024013.1; GI: 13128949. According to the present invention, human IFN α-1b encompasses the sequences shown in FIGS. 1A and 1B and described in Table 1, below, as well as any homologues, orthologs, variants, analogs, derivatives, active (e.g., biologically or pharmaceutically) fragments or mutants of IFN α-1b. For example, the IFN α-1b referred to herein may also be known in the art as leukocyte interferon, IFL, IFN, IFN α1, IFN alfa, and IFN-ALPHA. A comparison of a sequence of IFN α-1b (SEQ ID NOS. 1 and 2) with two IFN-α sequences described in the scientific literatures (Mantei et al, 1980, Gene 10: 1-10; Geoddel et al, 1981, Nature 390:20-26) given in Table 1. It is anticipated that the IFN-α sequences listed in Table 1 may be equally suitable for use in preparing the compositions of the present invention.

TABLE 1
IFN α1 Genes from Various Sources
Table 1. Amino Acid Variants of human IFN α1 Sequences from Various Sources

| | | Source (year of publication) | | | | |
|---|---|---|---|---|---|---|
| | | Mantei^{1,2} | Goeddel³ | Li⁴ | Ding⁵ | Chen⁶ |
| | Position | 1980 | 1981 | 1991 | 1996 | 2001 |
| Name in publication | | IFN α1 | IFN αD | IFN α1/158V | IFN α1b | IFN α1b |
| Name recommended by Li (3) | | IFN-α1b | IFN-α1a | IFN-α1c | — | — |
| Amino acid variant | 93 | Leu | Leu | Leu | Leu | Pro |
| | 100 | Val | Val | Ala | Ala | Val |
| | 114 | Ala | Val | Ala | Ala | Ala |
| | 149 | Met | Met | Met | Met | Val |
| | 158 | Leu | Leu | Val | Leu | Leu |

Note:
¹Mantei, N., Schwarzstein, M., Streuli, M., Panem, S., Nagata, S., and Weissmann, C.: The nucleotide sequence of a cloned human leukocyte interferon cDNA. (1980) Gene 10, 1-10
²Nagata, S., Mantei, N. and Weissmann, C., The structure of one of the eight or more distinct chromosomal genes for human interferon-α. (1980) Nature 287, 401-408.
³Goeddel, D. V., Leung, D. W., Dull, T. J., Gross, M., Lawn, R. M., McCandliss, R., Seeburg, P. H., Ullrich, A., Yelverton, E., and Gray, P. W.: The structure of eight distinct cloned human leukocyte interferon cDNAs. (1981) Nature 290, 20-26
⁴Li, M. F., Jin, Q., Hu, G., Guo, H. Y., and Hou, Y. D.: A novel variant of human interferon α1 gene. (1991) Science in China (Series B) 35, 200-206
⁵Ding, X. S., Human recombinant interferon α1b, Genetic Engineered Drugs (Chinese) (1996), 154-157
⁶Chen, H. H. and Yu, X. B.: Homo sapiens interferon alpha 1b gene, partial cds. Accession (AF439447), Version (AF439447, GI: 17063948), NCBI, submitted (24-OCT-2001), Sun Yat-Sen University of Medical Sciences, Guangzhou, Guangdong, P. R. China

IFN α-1b polynucleotides of the invention may comprise a native sequence (i.e., an endogenous sequence that encodes a IFN α-1b polypeptide or a portion thereof) or may comprise a variant, or a biological or antigenic functional equivalent of such a sequence. Polynucleotide variants may contain one or more substitutions, additions, deletions and/or insertions, as further described below, relative to a native polypeptide. The term “variants” also encompasses homologous genes of xenogenic origin. Typically, IFN α-1 b variants will retain all, a substantial proportion, or at least partial biological activity as, for example, can be determined using the interferon bioassay described below in Example 6, or the like. See also Rubinstein et al., J. Virol. 37:7551 (1981) which is incorporated by reference herein in its entirety.

Analogs of the IFN α-1b of the invention can be made by altering the protein sequences by substitutions, additions or deletions that provide for functionally equivalent molecules, as is well known in the art. These include altering sequences in which functionally equivalent amino acid residues are substituted for residues within the sequence resulting in a silent change. For example, one or more amino acid residues within the sequence can be substituted by another amino acid of a similar polarity, which acts as a functional equivalent, resulting e.g., in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. It is envisioned that both naturally occurring and genetically engineered (e.g., recombinant) variants containing conservative substitutions as well as those in regions of the protein that are not essential for biological activity will give functionally equivalent IFN α-1b polypeptides that are encompassed by the invention.

Also encompassed by the invention are fragments of IFN α-1b conjugated to polyol. As used herein, “fragments” of IFN α-1b refers to portions of IFN α-1b that are generated by any method, including but not limited to enzymatic digestion and chemical cleavage (e.g. CNBr) of IFN α-1 b and physical shearing of the polypeptide. Fragments of IFN α-1 b may also be generated, e.g. by recombinant DNA technology and by amino acid synthesis.

The polyol moiety in the polyol-IFN α-1b conjugate according to the present invention can be any water-soluble mono- or bifunctional poly(alkylene oxide) having a linear or branched chain. Typically, the polyol is a poly(alkylene glycol) such as poly(ethylene glycol) (PEG). However, those of skill in the art will recognize that other polyols, such as, for example poly(propylene glycol) and copolymers of polyethylene glycol and polypropylene glycol, can be suitably used.

Other interferon conjugates can be prepared by coupling an interferon to a water-soluble polymer. A non-limiting list of such polymers include other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used.

“PEG,” as used herein includes molecules of the general formula:
—CH₂CH₂O(CH₂CH₂O)ₙCH₂CH₂—
PEG includes linear polymers having hydroxyl groups at each terminus:

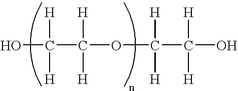

This formula can be represented in brief as HO-PEG-OH, where it is meant that -PEG- represents the polymer backbone without the terminal groups.

PEG is commonly used as methoxy-PEG-OH, (m-PEG), in which one terminus is the relatively inert methoxy group, while the other terminus is a hydroxyl group that is subject to chemical modification. The formula of methoxy PEG is shown below:
CH₃O—(CH₂CH₂O)ₙ—CH₂CH₂—OH

Branched PEGs are also in common use. The branched PEGs can be represented as R(-PEG-OH)ₘ in which R represents a central core moiety such as pentaerythritol, glycerol, or lysine and m represents the number of branching arms. The number of branching arms (m) can range from three to a hundred or more. The hydroxyl groups are further subject to chemical modification.

Another branched form, such as that described in PCT patent application WO 96/21469, has a single terminus that is subject to chemical modification. This type of PEG can be represented as (CH₃O-PEG-)ₚR—X, whereby p equals 2 or 3, R represents a central core such as lysine or glycerol, and X represents a functional group such as carboxyl that is subject to chemical activation. Yet another branched form, the “pendant PEG”, has reactive groups, such as carboxyl, along the PEG backbone rather than at the end of PEG chains.

In addition to these forms of PEG, the polymer can also be prepared with weak or degradable linkages in the backbone. For example, Harris has shown in U.S. patent application Ser. No. 06/026,716, which is incorporated by reference herein in its entirety, that PEG can be prepared with ester linkages in the polymer backbone that are subject to hydrolysis. This hydrolysis results in cleavage of the polymer into fragments of lower molecular weight, according to the reaction scheme:
-PEG-CO₂-PEG-+H₂O→-PEG-CO₂H+HO-PEG-

The term polyethylene glycol or PEG is meant to comprise native PEG as well as all the above described derivatives.

The copolymers of ethylene oxide and propylene oxide are closely related to PEG in their chemistry, and they can be used instead of PEG in many of its applications. They have the following general formula:
HO—CH₂CHRO(CH₂CHRO)ₙCH₂CHR—OH
wherein R is H or CH3, CH2CH3, (CH2)mCH3.

PEG is a useful polymer having the property of high water solubility as well as high solubility in many organic solvents. PEG is generally non-toxic and non-immunogenic. When PEG is chemically attached (“PEGylation”) to a water insoluble compound, the resulting conjugate generally becomes water soluble, as well as soluble in many organic solvents.

As used herein, the term “PEG moiety” is intended to include, but is not limited to, linear and branched PEG, methoxy PEG, hydrolytically or enzymatically degradable PEG, pendant PEG, dendrimer PEG, copolymers of PEG and one or more polyols, and copolymers of PEG and PLGA (poly(lactic/glycolic acid)). According to the present invention, the term polyethylene glycol or PEG is meant to comprise native PEG as well as all derivatives described herein.

“Salts” as used herein refers both to salts of the carboxyl-groups and to the salts of the amino functions of the compound obtainable through known methods. The salts of the carboxyl-groups include inorganic salts as, for example, sodium, potassium, calcium salts and salts with organic bases as those formed with an amine as triethanolamine, arginine or lysine. The salts of the amino groups included for example, salts with inorganic acids as hydrochloric acid and with organic acids as acetic acid.

“Functional derivatives” as herein used refers to derivatives which can be prepared from the functional groups present on the lateral chains of the amino acid moieties or on the terminal N— or C— groups according to known methods and are included in the present invention when they are pharmaceutically acceptable, i.e., when they do not destroy the protein activity or do not impart toxicity to the pharmaceutical compositions containing them. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups.

“Precursors” are compounds which are converted into IFN α-1b in the human or animal body.

As “active fractions” of the protein, the present invention refers to any fragment or precursor of the polypeptidic chain of the compound itself, alone or in combination with related molecules or residues bound to it, for example, residues of sugars or phosphates, or aggregates of the polypeptide molecule when such fragments or precursors show the same activity of IFN α-1b as medicament.

The conjugates of the present invention can be prepared by any of the methods known in the art. According to one embodiment of the invention, IFN α-1b is reacted with the PEGylating agent in a suitable solvent and the desired conjugate is isolated and purified, for example, by applying one or more chromatographic methods.

“‘Thiol-reactive PEGylating agent,” as used herein, means any PEG derivative which is capable of reacting with the thiol group of a cysteine residue. It can be, for example, PEG containing a functional group such as orthopyridyl disulfide, vinylsulfone, maleimide, iodoacetimide, and orthopyridyl disulfide (OPSS) derivatives of PEG. In one aspect of the invention, the PEGylating agent is a sulphydryl-selective PEG. In one embodiment of the invention the PEGylating agent is an mPEG-MAL, which can be represented by the formula:

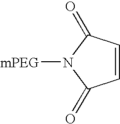

In another embodiment, the PEGylating agent is an mPEG2-MAL, which can be represented by the formula:

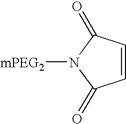

In preferred embodiments, the PEGylating agent is mPEG-MAL or mPEG2MAL from Nektar Therapeutics.

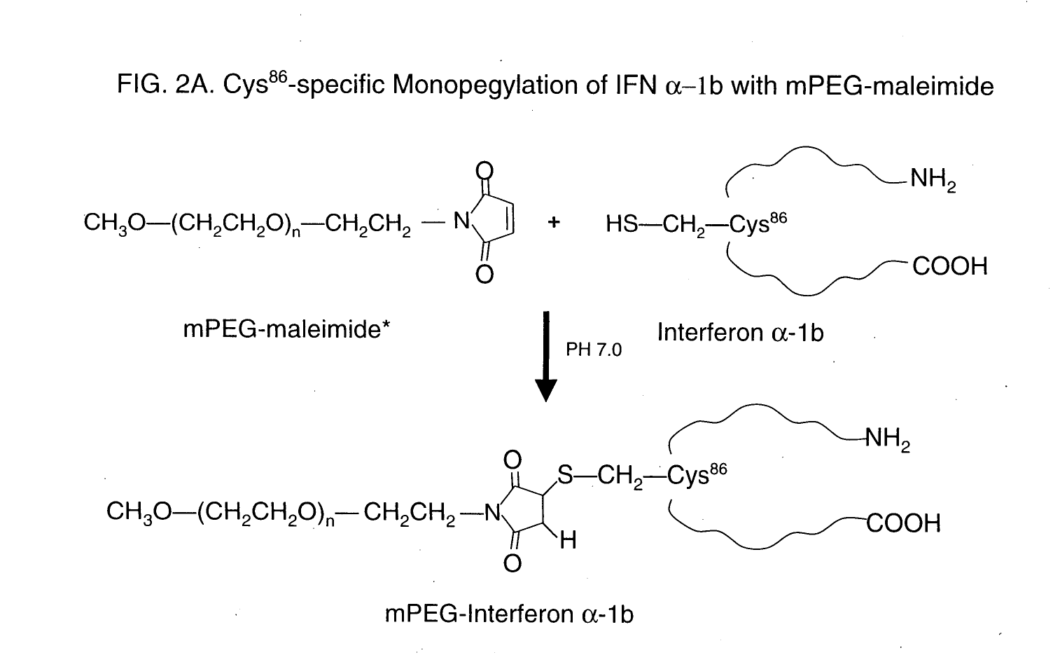

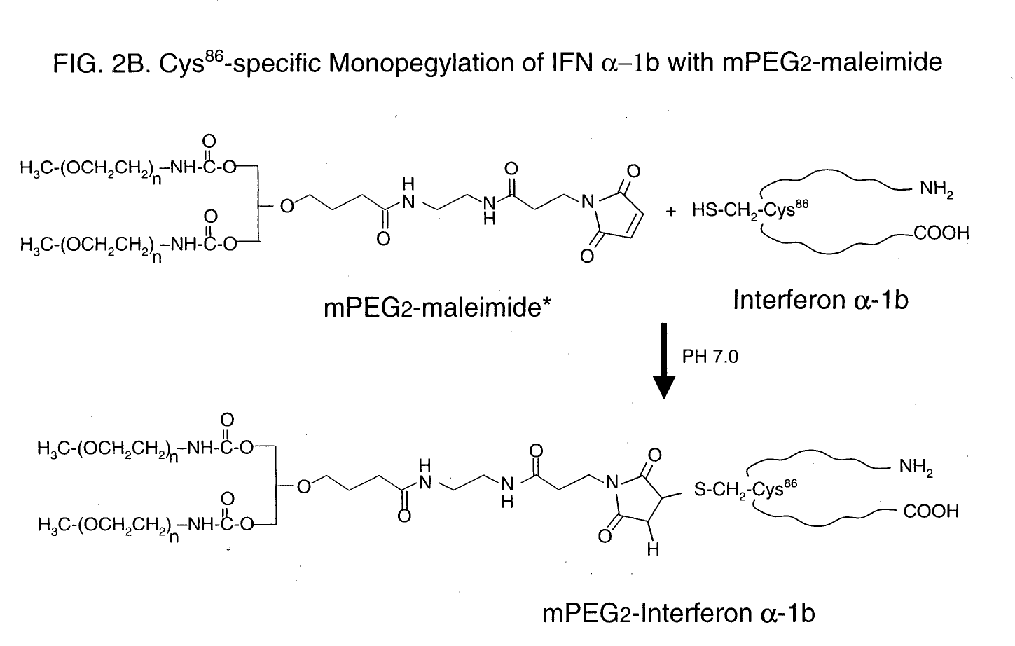

A typical reaction scheme for the preparation of the conjugates of the invention is presented in FIGS. 2A and 2B.

The type of thioether that is produced between a protein and PEG moieties has been shown to be stable in the circulation, but it can be reduced upon entering the cell environment. Without wishing to limit the present invention to any one theory or mode of action, in one embodiment of the invention, the conjugate, which does not enter the cell, is stable in the circulation until it is cleared.

It should be noted that the above reaction is site-specific for IFN α-1b because on the Cys at position 86 is available for interaction with the mPEG-MAL reagent; the other Cys residues appearing at amino acid positions 1, 29, 99, and 139 in the naturally occurring form of human IFN α-1b do not react with the PEGylating agent since they form disulfide bonds (i.e., Cys¹-Cys⁹⁹; Cys²⁹-Cys¹³⁹).

In certain embodiments, a polyol-interferon α-1b conjugate of the present invention has the same or higher interferon-α activity as native human interferon α-1b. In another embodiment, the polyol IFN α-1b has partial or substantial activity, as native human interferon α-1b. In other embodiments, the polyol IFN α-1 b has at least a measurable amount of activity. The comparative activity of conjugated and unconjugated interferon α-1b can be determined by any method available for determining interferon activity, such as measuring biological anti-viral, anti-inflammatory or anti-tumor properties in vitro or in vivo. In one assay suitable for used in the present invention, cytopathic effect inhibition is measured. See Rubinstein et al., J. Virol. 37:755 (1981). Interferon protects cells from viral infection (cytopathic effect) therefore increases the viability of host cells under viral infection. Thus, according to this method, interferon inhibits viral cytopathic effect (CPE) in host cells, which is measured by cell proliferation or viability.

The polyol-interferon α-1b conjugate will, in one aspect of the invention, have a homogenous molecular weight. The molecular weight can be determined by any means available in the art, including, but not limited to, native or denaturing gel electrophoresis, gel filtration, size exclusion chromatography, ultrafiltration and mass spectrometry.

“Chromatographic method” or “chromatography” refers to any technique that is used to separate the components of a mixture by their application on a support (stationary phase) through which a solvent (mobile phase) flows. The separation principles of the chromatography are based on the different physical nature of stationary and mobile phase.

Some particular types of chromatographic methods, which are well-known in the literature, include: liquid, high pressure liquid, ion exchange, absorption, affinity, partition, hydrophobic, reversed phase, gel filtration, ultrafiltration or thin-layer chromatography.

The PEGylating agent can be used in its mono-methoxylated form where only one terminus is available for conjugation, or in a bifunctional form where both termini are available for conjugation, such as for example in forming a conjugate with two IFN α-1b covalently attached to a single PEG moiety. The PEGylating agent typically has a molecular weight between 500 and 100,000.

The present invention is also directed to a method for the preparation of a polyol-interferon conjugate comprising the steps of providing an interferon with a single free cysteine group and a maleimide polyol or a maleimide bis-polyol, contacting the interferon with the maleimide polyol or with the maleimide bis-polyol under conditions which permit formation a covalent bond (i.e. thioether bond) between the polyol and the free cysteine at any position, thereby producing a polyol-interferon conjugate. According to this method, the interferon can be any interferon that has a single free cysteine. In one embodiment, the interferon is a naturally occurring protein that has a single free cysteine, but may contain additional cysteine that naturally form intramolecular disulfide bonds. In another embodiment, the interferon has been engineered, e.g., by recombinant DNA methodology, to have a single free cysteine, either by eliminating undesirable cysteines or by adding to or mutating the nucleotide sequence to encode a new cysteine. The interferons can also be engineered as fusion proteins or chimeric proteins wherein the two or more proteins are combined to take advantage of the desirable properties of multiple species, including, but not limited to, a free cysteine site for PEGylation. Methods for engineering the interferons of the present invention will be well known to those skilled in the art. See, for example, Sambrook et al. Molecular Cloning: A Laboratory Manual (2d ed., Cold Spring Harbor Laboratory Press, Cold Spring Harbor, N.Y. (1989)); Ausubel et al., Current Protocols in Molecular Biology (John Wiley & Sons Inc., N.Y. (2003)), the contents of which are incorporated by reference herein in their entirety.

In certain embodiments of this method of the present invention, the interferon is an alpha interferon, such as IFN α-1b, which contains a single free cysteine.

Furthermore, the general methodology is applicable to any protein that has an available sulphydryl residue. According to one aspect of the invention, the method is used to modify proteins, polypeptides and peptides that have a single sulphydryl residue, e.g., a single free cysteine residue. In another aspect, the proteins, polypeptides or peptides contain an number of cysteine residues such that each pair of cysteine residues form disulfide bonds and the remaining cysteine is free for modification using, e.g a mPEG-MAL or mPEG₂-MAL. Thus, according to this aspect of the invention, a protein, polypeptide or peptide comprising 3 cysteine residues would form a disulfide bonded pair, leaving a single free cysteine; while a 7 cysteine-containing species would form 3 disulfide bonded pairs with a single cysteine free for PEGylation.

The PEG-polypeptide conjugates of the present invention can be used to produce a medicament or pharmaceutical composition useful for treating diseases, conditions or disorders for which the polypeptides is effective as an active ingredient. Thus, the present invention also provides pharmaceutical compositions comprising a polyol-interferon-α conjugate having a polyol moiety covalently bound to Cys⁸⁶ of human interferon α-1 b, and a pharmaceutically acceptable carrier, excipient or auxiliary agent.

IFN α-1b conjugates of the present invention and pharmaceutically acceptable salts, solvates and hydrates thereof are expected to be effective in treating diseases or conditions that can be mediated by interferon α-1 b. Therefore, compounds of the invention and pharmaceutically acceptable salts, solvates and hydrates thereof are believed to be effective in inflammatory disorder, infections and cancer.

In one embodiment of the present invention substantially purified conjugates are provided in order for them to be suitable for use in pharmaceutical compositions, as active ingredient for the treatment, diagnosis or prognosis of bacterial and viral infections as well as autoimmune, inflammatory diseases and tumors. Non-limiting examples of the above-mentioned diseases include: septic shock, AIDS, rheumatoid arthritis, lupus erythematosus and multiple sclerosis.

The present invention also provides methods of modulating processes mediated by interferon-α comprising administering to a patient an effective amount of a polyol-interferon α-1b conjugate. Such process include, but are not limited to inflammation, viral infection, bacterial infection and cancer. In yet another embodiment the present invention provides a method of treating a patient with an interferon-α-responsive condition or disease, comprising administering to a patient an effective amount of a polyol-interferon α-1b conjugate. It is envisioned that this treatment may be useful for any disease or condition in which interferon therapy my provide a treatment, palliation, amelioration or the like, including without limitation inflammatory disorders (e.g., is multiple sclerosis, arthritis, asthma, cystic fibrosis, or interstitial lung disease); viral infections (e.g., hepatitis C infection, hepatitis B infection or HIV infection); bacterial infections well known in the art, particularly those refractory or resistant to conventional treatment with, e.g., antibiotics; and cancer (e.g., myeloma, lymphoma, liver cancer, breast cancer, melanoma, and hairy-cell leukemia).

An embodiment of the invention is the administration of a pharmacologically active amount of the conjugates of the invention to subjects at risk of developing e.g. one of the diseases listed above or to subjects already showing such pathologies.

Any route of administration compatible with the active principle can be used. Parenteral administration, such as subcutaneous, intramuscular or intravenous injection is preferred in certain embodiments of the invention. The dose of the active ingredient to be administered depends on the basis of the medical prescriptions according to age, weight and the individual response of the patient.

IFN α-1b conjugates of the present invention can be combined in a mixture with a pharmaceutically acceptable carrier to provide pharmaceutical compositions useful for treating the biological conditions or disorders noted herein in mammalian, and particularly in human patients. The particular carrier employed in these pharmaceutical compositions may take a wide variety of forms depending upon the type of administration desired. Suitable administration routes include enteral (e.g., oral), topical, suppository, inhalable and parenteral (e.g., intravenous, intramuscular and subcutaneous).

In preparing the compositions in oral liquid dosage forms (e.g., suspensions, elixirs and solutions), typical pharmaceutical media, such as water, glycols, oils, alcohols, flavoring agents, preservatives, coloring agents and the like can be employed. Similarly, when preparing oral solid dosage forms (e.g., powders, tablets and capsules), carriers such as starches, sugars, diluents, granulating agents, lubricants, binders, disintegrating agents and the like will be employed. Due to their ease of administration, tablets and capsules represent a desirable oral dosage form for the pharmaceutical compositions of the present invention.

The pharmaceutical composition for parenteral administration can be prepared in an injectable form comprising the active principle and a suitable vehicle. For parenteral administration, the carrier will typically comprise sterile water, although other ingredients that aid in solubility or serve as preservatives may also be included. Furthermore, injectable suspensions may also be prepared, in which case appropriate liquid carriers, suspending agents and the like will be employed. Vehicles for the parenteral administration are well known in the art and include, for example, water, saline solution, Ringer solution and/or dextrose. The vehicle can contain small amounts of excipients in order to maintain the stability and isotonicity of the pharmaceutical preparation. The preparation of the solutions can be carried out according to the ordinary modalities.

For topical administration, the IFN α-1b conjugates of the present invention may be formulated using bland, moisturizing bases, such as ointments or creams. Examples of suitable ointment bases are petrolatum, petrolatum plus volatile silicones, lanolin and water in oil emulsions such as Eucerin™, available from Beiersdorf (Cincinnati, Ohio). Examples of suitable cream bases are Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Warner-Lambert (Morris Plains, N.J.).

The pharmaceutical compositions and IFN α-1b conjugates of the present invention will generally be administered in the form of a dosage unit (e.g., liquid, tablet, capsule, etc.). The compounds of the present invention generally are administered in a daily, weekly, and monthly dosages of from about 0.01 μg/kg of body weight to about 50 mg/kg of body weight. Typically, the IFN α-1b conjugates of the present invention are administered in a daily, weekly, and monthly dosages of from about 0.1 μg/kg to about 25 mg/kg of body weight. Frequently, the compounds of the present invention are administered in a daily, weekly, and monthly dosages of from about 1 μg/kg to about 5 mg/kg body weight. The dosage can be between 10 μg and 1 mg daily for an average body weight of 75 kg, and in one embodiment the daily dose is between 20 μg and 200 μg. Furthermore, the extended action of the modified IFN α-1b conjugates may facilitate, e.g, a weekly, or biweekly dosing schedule. For example, the dosage can be about 10 to about 500 μg per person per week. In certain embodiments, the weekly dosage can be about 50 to about 250 μg per person. In other embodiments, the dosage can be about 100 to about 200 μg per person per week. As recognized by those skilled in the art, the particular quantity of pharmaceutical composition according to the present invention administered to a patient will depend upon a number of factors, including, without limitation, the biological activity desired, the condition of the patient, and tolerance for the drug.

The present invention has been described with reference to the specific embodiments, but the content of the description comprises all modifications and substitutions which can be brought by a person skilled in the art without extending beyond the meaning and purpose of the claims.

### EXAMPLES

The invention will now be described by means of the following Examples, which should not be construed as in any way limiting the present invention.

### Example 1

### Preparation of Recombinant Human Interferon α-1b

Recombinant human interferon α-1b (referred to as “IFN α-1b” or “rhIFN α-1b”) was prepared by fermentation of an E. coli engineered to express the IFN α-1b DNA sequence shown in FIG. 1 (SEQ. ID No.s 1, 2, and 3). The fermented cells were harvested and centrifuged to form cell pastes. The IFN α-1b was then purified by ion exchange, affinity, and size-exclusion chromatography. IFN α-1b may also be obtained from commercial sources. In certain experiments, the IFN α-1b was provided by Shenzhen Kexing Bio-product Co. (Shenzhen, China).

### Example 2

### Preparation of mPEG (20 kD)-IFN α-1b

IFN α-1b was conjugated with an activated N-maleimide derivative of a single chain methoxy polyethylene glycol (mPEG (20 kD)-MAL) (Nektar Therapeutics, Huntsville, Ala.). The PEG polymer had an average molecular weight of 21.5 kD.

Conjugation of IFN α-1b with a Single Chain mPEG (20 kD)-Maleimide

One gram of IFN α-1b was diafiltered into 50 mM sodium phosphate buffer, pH 7.0, using an Amicon Ultrafiltration Cell (350 mL) with YM-10 membrane (Millipore, Bedford, Mass.). The concentration of IFN α-1b was finally diluted to approximately 1 mg/mL. mPEG (20 kD)-MAL was added in a molar excess of 3 moles to one mole of IFN α-1b and the solution was gently stirred for 2 hours at room temperature. The reaction was monitored by SDS-PAGE to determine the extent of conjugation. Under these conditions, the free sulfhydryl group of cysteine at position 86 on IFN α-1b was specifically linked via a stable thioether bond to the activated maleimide group of mPEG (20 kD)-MAL. The molecular structure of mPEG (20 kD)-MAL and Cys-specific conjugation mechanism are illustrated in FIG. 2A.

The final products of conjugation contained predominantly mono-pegylated IFN α-1b, high molecular weight species, unconjugated IFN α-1b, and mPEG (20 kD)-MAL.

Purification of mPEG (20 kD)-IFN α-1b

Hydrophobic interaction chromatography (HIC) was used to separate mPEG (20 kD)-IFN α-1b from unconjugated IFN α-1b and mPEG (20 kD)-MAL as follows. Sodium citrate was added to the post-conjugation solution to reach a final concentration of 0.4 M. The solution was loaded onto a Butyl Sepharose™ 4 Fast Flow (GE Healthcare, New Jersey) column (5.0 cm×13.5 cm; bed volume of 265 mL) equilibrated with Buffer A (0.4 M sodium citrate in 50 mM Tris, pH 6.8). The column was washed with 5 column volumes of Buffer A to remove unconjugated IFN α-1b and mPEG (20 kD)-MAL. The mono-pegylated mPEG (20 kD)-IFN α-1b was eluted using a linear gradient from 0-50% of Buffer B (50 mM Tris, pH 6.8) over 10 column volumes. The protein content of the eluent was monitored at 280 nm. The column was eluted at a flow rate of 30 ml/min, and the mPEG (20 kD)-IFN α-1b fractions were collected and pooled for a total volume of 1150 mL of pooled mPEG (20 kD)-IFN α-1b.

Size exclusion chromatography was used to separate mono-pegylated IFN α-1b from high molecular weight species. The pooled fractions from HIC were diafiltered into Buffer C (20 mM sodium acetate/0.14 M sodium chloride, pH 6.0) and concentrated to 6-8 mg/mL. The concentrated solution was then loaded onto a Superdex™ 75 (GE Healthcare, New Jersey) column (16×53 cm; 106 mL bed volume) pre-equilibrated in Buffer C. The mono-pegylated IFN α-1b was eluted by Buffer C at a flow rate of 1 ml/min. The protein content of the eluent was monitored at 280 nm. The mPEG (20 kD)-IFN α-1b fractions were collected and pooled for a total of 20 mL. Approximately 0.2 gram of mono-pegylated IFN α-1b was obtained after the conjugation and purification, representing an overall yield of approximately 20%.

### Example 3

### Preparation of mPEG₂ (40 kD)-IFN α-1b

IFN α-1b was conjugated with an activated N-maleimide derivative of a branched chain methoxy polyethylene glycol {maleimidopropionamide of bis [(methoxy poly (ethylene glycol) average MW 40,000], modified glycerol} (mPEG2 (40 kD)-MAL) (Nektar Therapeutics, Huntsville, Ala.) as described above in Example 2 for mPEG (20 kD)-IFN α-1b. The PEG polymer had an average molecular weight of 42.4 kD. The molecular structure of mPEG2 (40 kD)-MAL and Cys-specific conjugation mechanism are illustrated in FIG. 2B. Purification of mPEG2 (40 kD)-IFN α-1b was as described in Example 2.

### Example 4

### Characterization of mPEG-IFN α-1b Conjugates

mPEG (20 kD)-IFN α-1b and mPEG₂ (40 kD)-IFN α-1b were characterized as described below to determine the purity and molecular weights of the conjugates.

SDS-PAGE Analysis

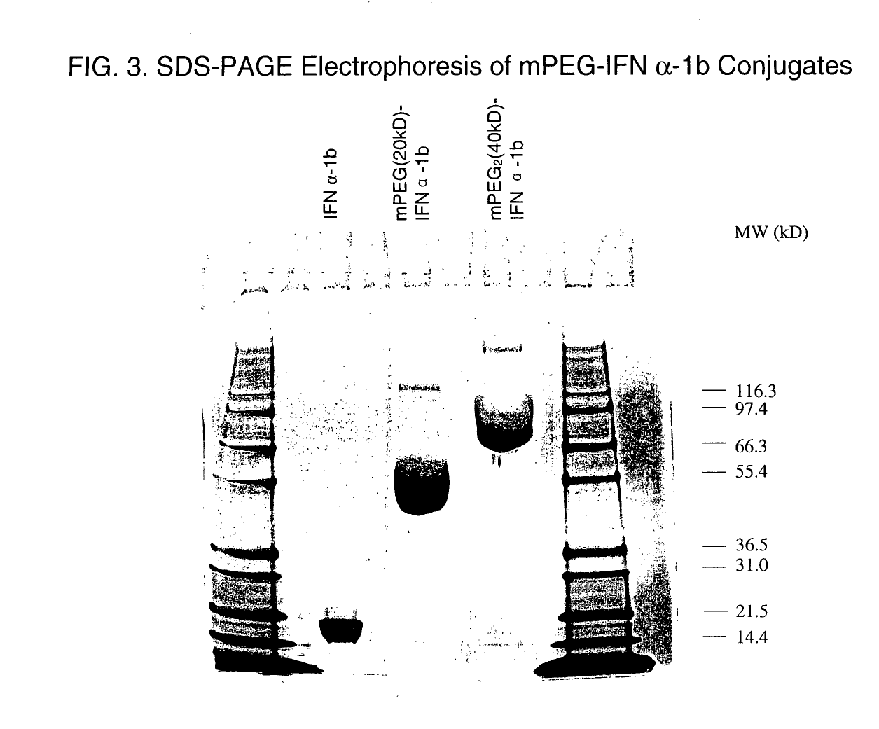

The molecular weight of unconjugated IFN α-1b, mPEG (20 kD)-IFN α-1b, and mPEG₂ (40 kD)-IFN α-1b were determined by SDS-PAGE gel electrophoresis. Samples equivalent of 10 μg unmodified IFN α-1b were loaded onto 4-12% BisTris NuPage gels (Invitrogen, California) according to the method of Laemmli (Nature 227:680-685 (1970)) and visualized by Coomassie Blue staining. As shown in FIG. 3, the apparent molecular weights of mPEG (20 kD)-IFN α-1b and mPEG₂ (40 kD)-IFN α-1b were 49.7 kD and 74.6 kD, respectively. The apparent molecular sizes of mPEG-IFN α-1b conjugates during polyacrylamide gel electrophoresis were significantly increased (as compared to unmodified globular IFN α-1b protein) by the attachment of long, linear PEG polymer chains.

SEC-HPLC Analysis

The purified mPEG-IFN α-1b conjugates were analyzed by size exclusion-high performance liquid chromatography (SEC-HPLC), using a Hewlett-Packard Series 1100 analytical HPLC system equipped with a Superose™ 12 HR (GE Healthcare, New Jersey) column (10×300 mm; particle size 10 μm). The mobile phase was 0.1 M sodium phosphate/0.15 M sodium chloride, pH 6.0, and the flow rate was 0.5 mL/min. The signals were monitored at 214 nm.

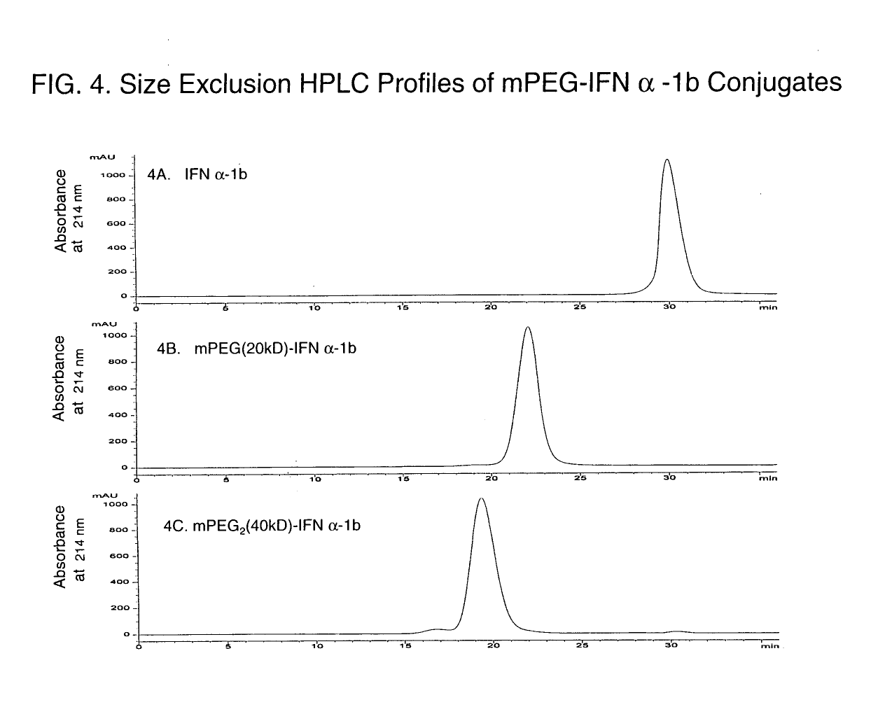

As shown in FIGS. 4A-C, mPEG-IFN α-1b conjugates were separated from IFN α-1b and high molecular weight species. The apparent molecular weights of mPEG (20 kD)-IFN α-1b and mPEG₂ (40 kD)-IFN α-1b were measured at 312 kD and 769 kD, respectively. The hydrodynamic volumes of mPEG-IFN α-1b conjugates observed during size exclusion chromatography were significantly increased (as compared to a globular IFN α-1b protein) by the attachment of long linear PEG polymer chains. The purities of mPEG (20 kD)-IFN α-1b and mPEG₂ (40 kD)-IFN α-1b were determined at 98.9% and 96.8%, respectively.

Mass Spectrometry

The molecular weights of mPEG-IFN α-1b conjugates were determined by matrix-assisted laser desorption/ionization (MALDI)-time-of-flight mass spectrometry performed on an Applied Biosystems Voyager-DE mass spectrometer with delayed extraction. Samples, deposited on the sample plate with sinapinic acid matrix, were irradiated with a nitrogen laser (Laser Science Inc., Massachusetts) operated at 337 nm. The laser beam was attenuated by a variable attenuator and focused on the sample target. Ions produced in the ion source were accelerated with a deflection voltage of 25,000 V. The ions were then differentiated according to their m/z using a time-of-flight mass analyzer.

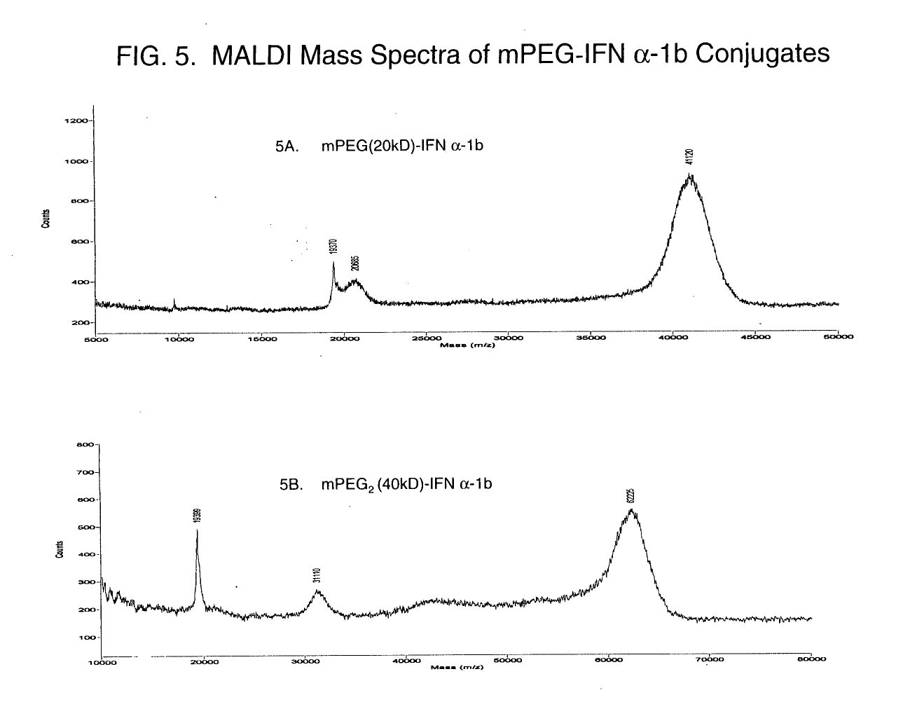

FIG. 5A shows the major peak of mPEG (20 kD)-IFN α-1b (41.1 kD) that was observed. The smaller 20.6 kD peak represented the same monopegylated IFN α-1b, which was charged with 2H+. The 19.4 kD peak represented residual IFN α-1b present in the sample.

FIG. 5B shows the major peak of mPEG₂ (40 kD)-IFN α-1b (62.2 kD) that was observed. The smaller 31.1 kD peak represented the same monopegylated IFN α-1b, which was charged with 2H+. The 19.4 kD peak represented residual IFN α-1b present in the sample.

The molecular weights of MPEG-IFN α-1b conjugates were determined by different methods are summarized in Table 2.

TABLE 2
Molecular Weights of Pegylated IFN α-lb Conjugates

| | | mPEG (20 kD)- | mPEG₂ (40 kD)- |
|---|---|---|---|
| | IFN α-lb | IFN α-lb | IFN α-lb |
| | MW (kD) | MW (kD) | MW (kD) |
| PEG | — | 21.5 | 42.4 |
| Expected (calculated) | 19.4 | 40.9 | 61.8 |
| MALDI-MS | 19.4 | 41.1 | 62.2 |
| (Absolute) | | | |
| SDS-PAGE | 18.4 | 49.7 | 74.6 |
| (Apparent) | | | |
| SEC-HPLC (Apparent) | 21.5 | 312 | 769 |

CEX-HPLC Analysis

The purified mPEG-IFN α-1b conjugates were analyzed by a modification of the high-performance cation exchange chromatography method of Monkarsh et al. (Anal. Biochem. 247:434-440 (1997) which is incorporated by reference herein in its entirety), using a Hewlett-Packard Series 1100 analytical HPLC system equipped with a TSK-GEL SP-5PW (Tosoh Biosciences, Pennsylvania) HPLC column (7.5×75 mm, 10 □m). The column was pre-equilibrated with at least 10 column volumes of Buffer A (5 mM sodium acetate, pH 4.1). mPEG-IFN α-1b conjugates were applied, and eluted at a flow rate of 0.6 mL/min by a linear ascending pH gradient (4.1 to 5.9) of 0% to 100% Buffer B (0.1 M sodium phosphate at pH 5.9) over 120 min. The proteins were monitored by absorbance at 214 nm.

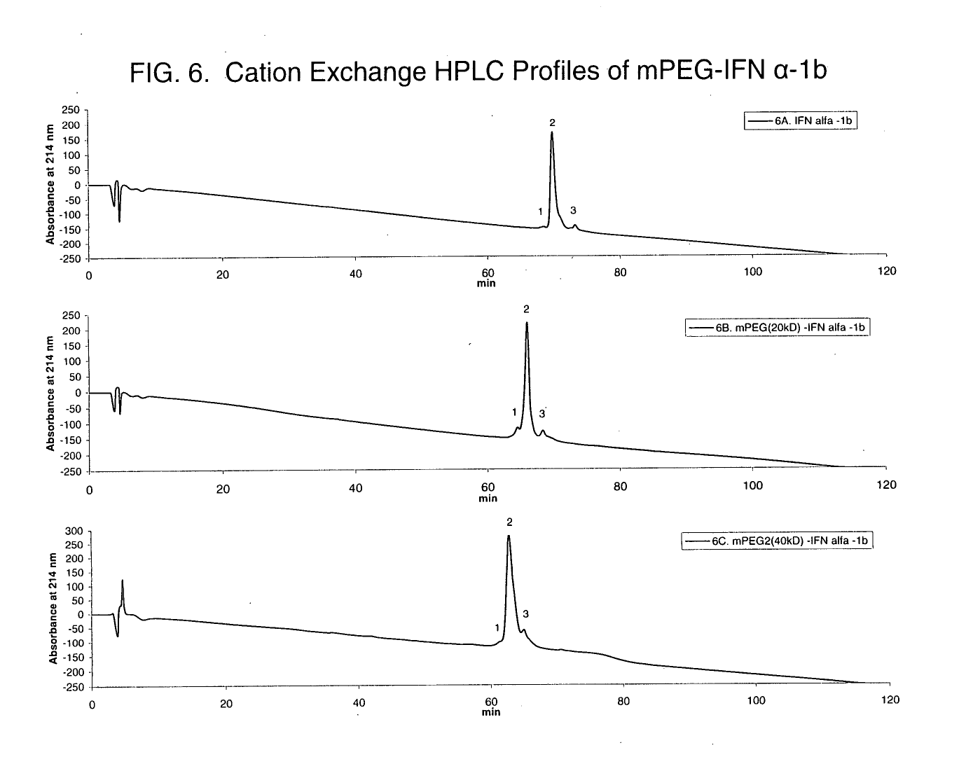

As shown in FIG. 6A, unmodified IFN α-1b (Peak 2) represented more than 92% of the sample applied. The identities of Peaks 1 and 3 were not determined.

As shown in FIG. 6B, mPEG (20 kD)-IFN α-1b (Peak 2) represented more than 90% of the sample applied. The identities of Peaks 1 and 3 were not determined. These results confirm that the maleimide group of mPEG-MAL was conjugated specifically to the free sulfhydryl group of residue Cys ⁸⁶ on IFN α-1b. No multiple positional isomers were observed.

As shown in FIG. 6C, mPEG2 (40 kD)-IFN α-1b (Peak 2) represented more than 87% of the sample applied. The identities of Peaks 1 and 3 were not determined. These results confirm that the maleimide group of mPEG2-MAL was conjugated specifically to the free sulfhydryl group of residue Cys⁸⁶ on interferon α-1b. No multiple positional isomers were observed.

### Example 5

### Characterization of Cys⁸⁶-Specific Mono-Pegylation of mPEG (20 kD))-IFN α-1b

Overview

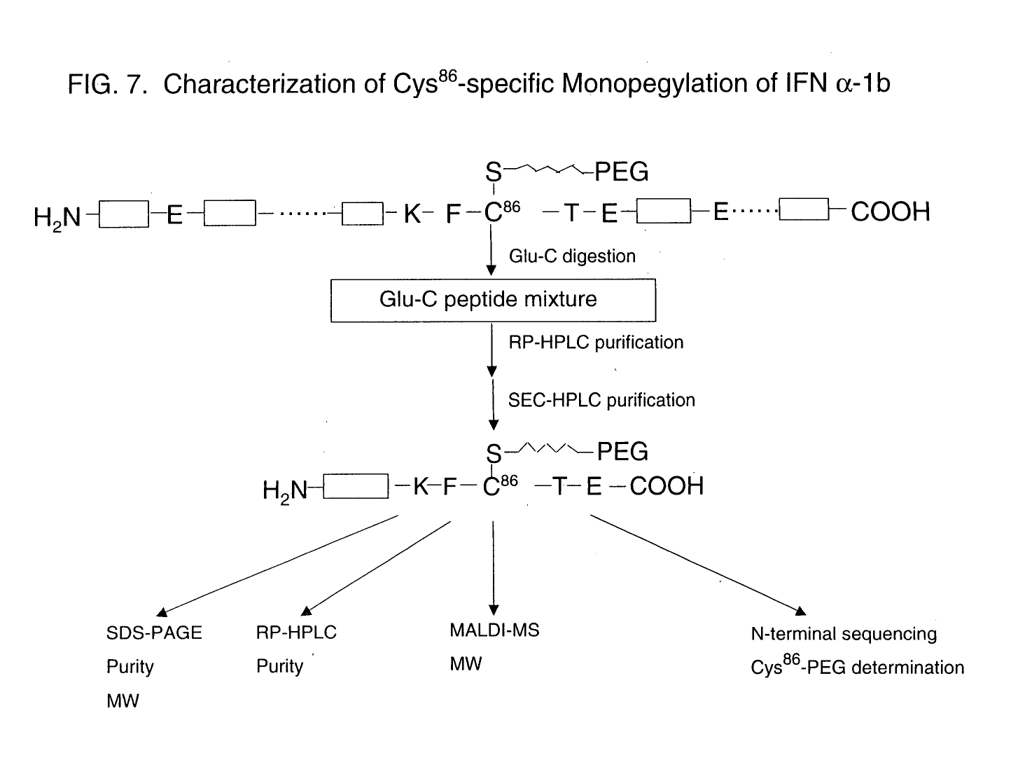

mPEG (20 kD)-IFN α-1b, reduced with dithiothreitol (DTT), was S-carboxymethylated by idoacetic acid. The S-carboxymethylated mPEG (20 kD)-IFN α-1b was digested by endoproteinase Glu-C, which was selected to generate 5 single-Cys-containing peptides and other non-Cys-containing peptides. FIG. 7 shows confirmation of Cys⁸⁶-specific mono-pegylation of IFN α-1b with mPEG (20 kD)-maleimide by peptide-mapping with endoproteinase Glu-C and by N-terminally sequencing a Cys⁸⁶-pegylated peptide isolated from Glu-C digests. The isolated Cys⁸⁶-pegylated peptide was analyzed for purity by reverse phase and size exclusion HPLC and for the molecular weight by SDS-PAGE and MALDI-MS. The Cys⁸⁶ residue of the isolated peptide was confirmed to be pegylated finally by N-terminal peptide sequencing.

Reductive Alkylation and Digestion by Endoproteinase Glu-C

5 mg of mPEG (20 kD)-IFN α-1b and 5 mg of the IFN α-1b reference were buffer-exchanged to a concentration of 1 mg/mL in 0.3 M Tris-HCl/6 M Guanidinum/1 mM EDTA, pH 8.4. DTT was added to reduce the disulfide bonds of IFN α-1b. Iodoacetic acid was added and the solution incubated at 37° C. for 20 minutes to S-carboxymethylate free sulfhydryl groups. The sample was buffer exchanged with 50 mM Ammonium Bicarbonate, pH 7.8 (digestion buffer). S-carboxymethylated mPEG (20 kD)-IFN α-1b was cleaved by endoproteinase Glu-C with an enzyme-to-protein ratio of 1:10 (w/w) in the digestion buffer at 25° C.

Peptide Mapping

The endoproteinase Glu-C digestion mixture was analyzed by reverse phase HPLC, using a Hewlett-Packard Series 1100 analytical HPLC system equipped with a C8-HPLC (Vydac, California) column (4.6×250 mm, 5 μm). Peptides were monitored by absorbance at 214 nm. Mobile phase A (H₂O/0.1% TFA) and mobile phase B (10% H₂O/90% Acetonitrile/0.1% TF) were used in a sectional gradient system for the separation of peptides:

Time (min)

| | 0 | 70 | 80 | 82 | 85 | 100 |
|---|---|---|---|---|---|---|
| B % | 0 | 80 | 92 | 92 | 0 | 0 |

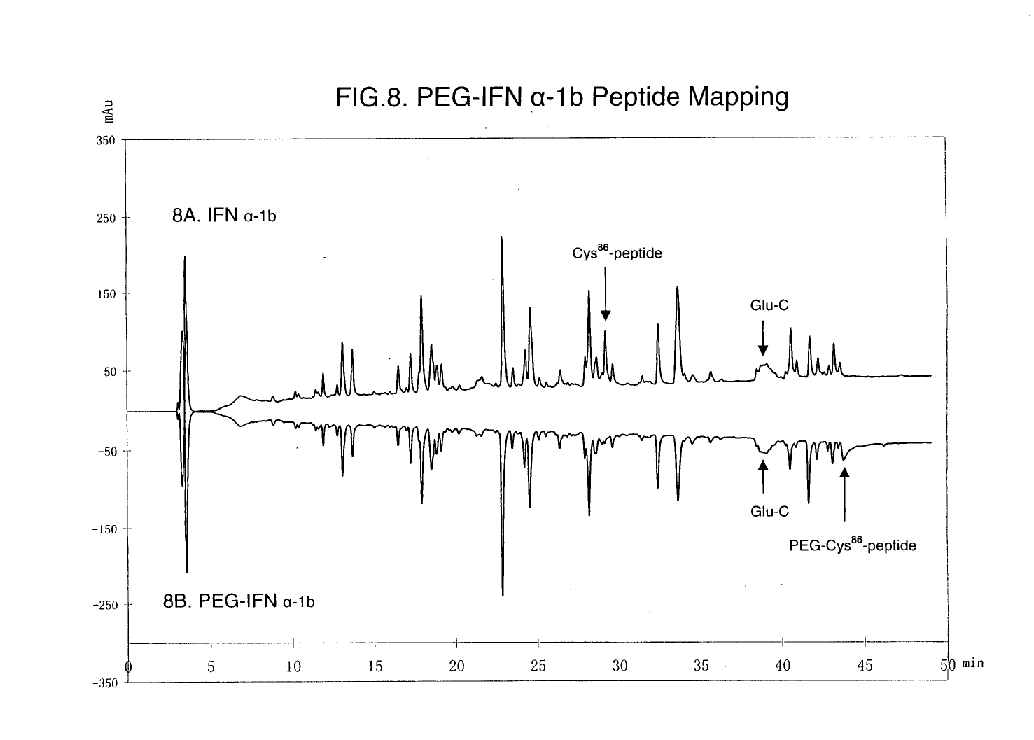

Peptide mapping fingerprints of unmodified IFN α-1b reference (FIG. 8A) and mPEG (20 kD)-IFN α-1b (FIG. 8B) were compared for the disappearance of an unmodified Cys⁸⁶-containing peptide and the appearance of a Cys⁸⁶-pegylated peptide. As shown in FIG. 8A, a peak at 29.1 minutes was observed, corresponding to the unmodified Cys 86-containing peptide. As shown in FIG. 8B, while the peak at 29.1 minutes disappeared, a new peak appearing at 43.7 minutes (similar with the retention time of mPEG (20 kD)-MAL in a separate experiment) was determined to be a pegylated peptide. The retention time increased from 29.1 minutes for Cys⁸⁶-containing peptide to 43.7 minutes for Cys⁸⁶-pegylated peptide was attributed principally by the attachment of large non-polar PEG polymers. The PEG polymers substantially reduced the polarity of a small Cys⁸⁶-containing peptide. These were the only significant differences observed in the peptide mapping fingerprints, indicating that a single Cys⁸⁶ residue was pegylated.

Isolation of Cys⁸⁶-Pegylated Peptide

The Cys⁸⁶-pegylated peptide (43.7-minute peak) was isolated by reverse phase C8-HPLC chromatography, as described above, from the endoproteinase Glu-C digests and further purified by size exclusion HPLC chromatography using a Superose™ 12 HR column (GE Healthcare, New Jersey). The Cys⁸⁶-pegylated peptide was confirmed by measuring its molecular weight using SDS-PAGE and MALDI mass spectroscopy. The purity of the Cys 86-pegylated peptide was determined by SDS-PAGE, reverse phase and size exclusion HPLC chromatography before proceeding to N-terminal peptide sequencing.

N-Terminal Peptide Sequencing

The Cys⁸⁶-pegylated peptide, isolated from the above peptide mapping, was N-terminally sequenced to determine its amino acid sequence by the Edman procedure (Edman, Acta Chem. Scand. 4:283 (1950), incorporated by reference herein in its entirety) using an ABI Procise® 494 Sequencer. The instrument delivered precise volumes of reagents to a cartridge where the polypeptide was immobilized on a PVDF membrane. At each cycle, the PTH-amino acid was transferred to the HPLC for analysis and quantification.

The peptide was sequenced for 16 cycles. The peptide sequence was detected:

H₂N-Ser⁷³-Ser⁷⁴-Ala⁷⁵-Ala⁷⁶-Trp⁷⁷-Asp⁷⁸-Glu⁷⁹-Asp⁸⁰-Leu⁸¹-Leu⁸²-Asp⁸³-
Lys⁸⁴-Phe⁸⁵-Cys⁸⁶-Thr⁸⁷-Glu⁸⁸-COOH

| | Cycle: | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| Detected: | + | + | + | + | + | + | + | + | + | + | − | + | + | − | + |

At the 14^{th} cycle, Cys⁸⁶ was not detected, indicating Cys⁸⁶ was pegylated at position 86.

Under digestion conditions used, Glu-C cleaved at Asp⁷² and at Glu⁸⁸ residues on the interferon molecule, generating the Ser⁷³-Glu⁸⁸ containing peptide being pegylated.

It is also recognized that long linear PEG polymer molecules attached on a protein may shield Glu⁷⁹ residues on the interferon protein from being cleaved by endoproteinase Glu-C.

### Example 6

### In Vitro Anti-Viral Activities of mPEG-IFN α-1b Conjugates by the WISH-VSV Cytopathicity Assay

The in vitro anti-viral activities of IFN α-1b and pegylated IFN α-1b conjugates were determined by the cytopathicity effect assay using WISH cells challenged by vesicular stomatitis virus (VSV) (Rubinstein et al., J. Virol. 37:755, 1981). The materials used in this WISH-VSV assay included WISH cells (ATCC, Rockville, Md.), VSV virus (ATCC, Rockville, Md.), IFN α-1b and the pegylated IFN α-1b conjugates prepared by the methods as described in Examples 1, 2, and 3

Serial two-fold dilutions of interferon samples were prepared in growth medium (DMEM, 2 mM L-glutamine, 10% FBS) in microtiter plates. The wells were seeded with 2.5×10⁴ WISH cells, and incubated at 37° C., 5% CO₂ for 18-24 hours. The cells were then infected with 10⁷ pfu of Vesicular Stomatitis Virus and incubated at 37° C. for an additional 24 hours. The assay samples were analyzed to determine cell proliferation using a colorimetric XTT assay (Roche Applied Science, Indiana).

The anti-viral activity of interferon was defined as the concentration (mg/mL) of interferon required to obtain 50% inhibition (IC₅₀) of the cytopathic effect. The specific activities of the interferon samples were calculated by comparing with IC₅₀ values of interferon samples with IC₅₀ of IFN α-2b (WHO) as an internal reference standard according to the equation:

$$\frac{{IC}_{50} Reference Standard ( U \text{/} ml )}{{IC}_{50} sample ( mg \text{/} ml )}$$

The results from the in vitro anti-viral assay are given below in Table 4.

TABLE 4
In Vitro Anti-viral Activities of IFN α-lb and mPEG-IFN α-lb
Conjugates*

| | Specific Activity | Residual Activity |
|---|---|---|
| | (IU/mg) | (%) of IFN α-lb |
| IFN α-lb | 1.11 ± 0.27 × 10⁷ (n = 4) | 100 |
| mPEG (20 kD)-IFN α-lb | 1.73 ± 0.25 × 10⁵ (n = 3) | 1.6 |
| mPEG₂ (40 kD)-IFN α-lb | 2.40 ± 0.29 × 10⁵ (n = 3) | 2.2 |

mPEG (20 kD)-IFN α-1b had approximately 1.6% residual IFN α-1b activity. mPEG₂ (40 kD)-IFN α-1b had approximately 2.2% residual IFN α-1b activity. SEC-HPLC analysis results (Example 4) indicated a relative higher content of unmodified IFN α-1b in the mPEG₂ (40 kD)-IFN α-1b preparation. The reduced anti-viral activity of pegylated interferon in this cytopathicity effect assay may be the result of the attachment of PEG polymers with their wrapping around the interferon molecule, thereby preventing ligand/receptor interaction of interferon with WISH cells. The In vitro activity of pegylated interferon is not necessarily reflective of in vivo pharmacological activity, however, as the PEG moieties may be removed from the interferon in the circulation, thereby revealing a more active form of the molecule. Without wishing to limit the invention to one theory or mode of action, the same mechanism that leads to increased stability of the pegylated interferon in vivo (see Example 7, below) may be responsible for the low level of activity observed in vitro. The reduced in vitro biological activity in the WISH assay was also observed with other pegylated interferon products such as pegylated IFN α-2a (see e.g., Bailon et al, Bioconjugate Chem. 12:195-202 (2001)) and pegylated IFN α-2b (see e.g., Wang et al, Advance Drug Delivery Rev., 54:547-570 (2002)).

### Example 7

### Pharmacokinetic Studies on Rats

Pharmacokinetic parameters of unmodified IFN α-1b, mPEG (20 kD)-IFN α-1b and mPEG₂ (40 kD)-IFN α-1b conjugates prepared by the methods described above were determined by implementing the pharmacokinetic protocol shown in Table 5.

TABLE 5
Protocol for Evaluation of Pharmacokinetic Parameters

| | IFN α-lb | mPEG (20 kD)-IFN α-lb | mPEG₂ (40 kD)-IFN α-lb |
|---|---|---|---|
| Rats | 6 | 6 | 6 |
| Dose (IFN protein | 208 | 1000 | 1000 |
| μg/Kg) | | | |
| Route | subcutaneous (S.C.) | subcutaneous (S.C.) | subcutaneous (S.C.) |
| Administration | single | single | single |
| Time points (hr) | 0.08, 0.17, 0.5, 0.75, 1, | 0.5, 2, 8, 12, 24, 48, 72, 96, | 0.5, 2, 8, 12, 24, 48, 72, |
| | 1.5, 2, 3, 4, 8, 12 | 120, 144, 168 | 96, 120, 144, 168 |

| | |
|---|---|
| Assay | ELISA immunoassay to quantitate IFN α-lb in rat serum at various time points. |

Each of 6 rats (control group) was subcutaneously injected with 208 μg of IFN α-1b/Kg body weight. Each of 6 rats of the two test groups was injected s.c. with a 1000 μg dose (protein equivalent of the IFN α-1b dose) of mPEG-IFN α-1b conjugate/Kg body weight. After a single subcutaneous administration of the test protein, blood samples were collected from the venous plexus of rat tails at each of 11 time points. The serum samples were separated from the whole blood by microcentrafigation and stored in frozen at −80° C. until all samples were collected. Interferon alpha in serum was quantitatively determined using a human interferon α-specific ELISA sandwich immunoassay (PBL Biomedical Laboratories, Piscataway, N.J.). The immunoassay demonstrates no cross-reactivity with rat IFN-α.

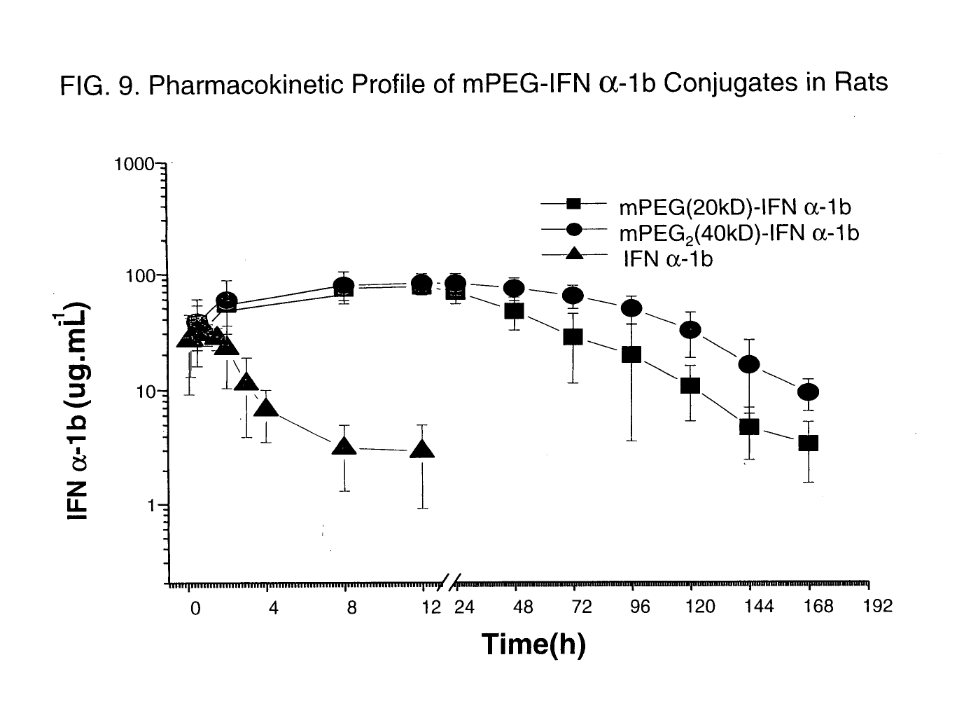

The pharmacokinetic profiles of mPEG-IFN α-1b conjugates are shown in FIG. 9 and major pharmacokinetic data are summarized in Table 6.

TABLE 6
Pharmacokinetic Parameters of mPEG-IFN α-lb on Rats Following Single S.C.
Administration

| | | Mean Value | | |
|---|---|---|---|---|
| Parameter | Unit | IFN α-lb | mPEG (20 kD)-IFN α-lb | mPEG₂ (40 kD)-IFN α-lb |
| PEG-conjugated | MW | — | 20 kD (single chain) | 40 kD (branched chain) |
| AUC₍₀₋ₜ₎ | μg · h · mL⁻¹ | 113.9 | 5135.7 | 8527.3 |
| Cₘₐₓ | μg · mL⁻¹ | 36.9 | 82.7 | 88.4 |
| Tₘₐₓ | h | 0.7 | 14.7 | 19.3 |
| t_{1/2(β)} | h | 3.4 | 30.9 | 30.7 |
| MRT | h | 3.0 | 45.5 | 61.3 |
| CL/F | mL · h⁻¹ · kg⁻¹ | 1.7 | 0.2 | 0.1 |

The major pharmacokinetic parameters of both mPEG (20 kD)-IFN α-1b and mPEG₂ (40 kD)-IFN α-1b conjugates were substantially different from those observed for with unmodified IFN α-1b. The area under the curve (AUC) was increased by 45-fold for mPEG (20 kD)-IFN α-1b and by 75-fold for mPEG₂ (40 kD)-IFN α-1b, compared to the AUC of unmodified IFN α-1b. Tₘₐₓ was increased by 20-fold for mPEG (20 kD)-IFN α-1b and by 25-fold for mPEG₂ (40 kD)—IFN α-1b, compared to the Tₘₐₓ of unmodified IFN α-1b. T_{1/2(β)} was increased by 9-fold for both of the mPEG-IFN α-1b conjugates.

There were no statistically significant differences in the values of Tₘₐₓ and T_{1/2(β)} between mPEG (20 kD)-IFN α-1b and mPEG₂ (40 kD)-IFN α-1b conjugates. However, the values of AUC, MRT and CL/F of mPEG₂ (40 kD)-IFN α-1b were significantly higher than those of mPEG (20 kD)-IFN α-1b.

### Example 8

### In Vivo Anti-Tumor Activity of mPEG-IFN α-1b

The in vivo anti-tumor properties of mPEG (20 kD)-IFN α-1b and interferon α-1b were determined on the inhibition of tumor growth on mice implanted with human tumor cells. Athymic Balb/C nude mice received a subcutaneous implant of 2×10⁶ human renal tumor ACHN cells (ATCC, Rockville, Md.). Three weeks were allowed for the tumors to get established. Mice were injected subcutaneously in the contralateral flank once weekly (Monday) with each of the dosages of 50 μg, 150 μg, and 300 μg of mPEG (20 kD)-IFN α-1b or thrice weekly (Monday, Wednesday, and Friday) with 50 μg of IFN α-1b (Table 7). The mice were treated for five weeks. Tumor volumes were measured every Monday prior to treatments.

TABLE 7
Evaluation of In Vivo Anti-tumor Activity and Measurement
of Tumor Volme

| | | | | | Tumor |
|---|---|---|---|---|---|
| | | | Dose/ | Injection | Volume |
| | | | mouse (IFN | (s.c.)/ | (cm³) in |
| Group | Testing drug | Mice | protein μg) | Wk | 5 wks |
| 1 | Placebo | 6 | — | 1 | 1.00 ± 0.37 |
| 2 | PEG-IFN α-1b | 6 | 50 | 1 | 0.46 ± 0.30 |
| 3 | PEG-IFN α-1b | 6 | 150 | 1 | 0.36 ± 0.13 |
| 4 | PEG-IFN α-1b | 6 | 300 | 1 | 0.27 ± 0.13 |
| 5 | IFN α-1b | 6 | 50 | 3 | 0.39 ± 0.07 |

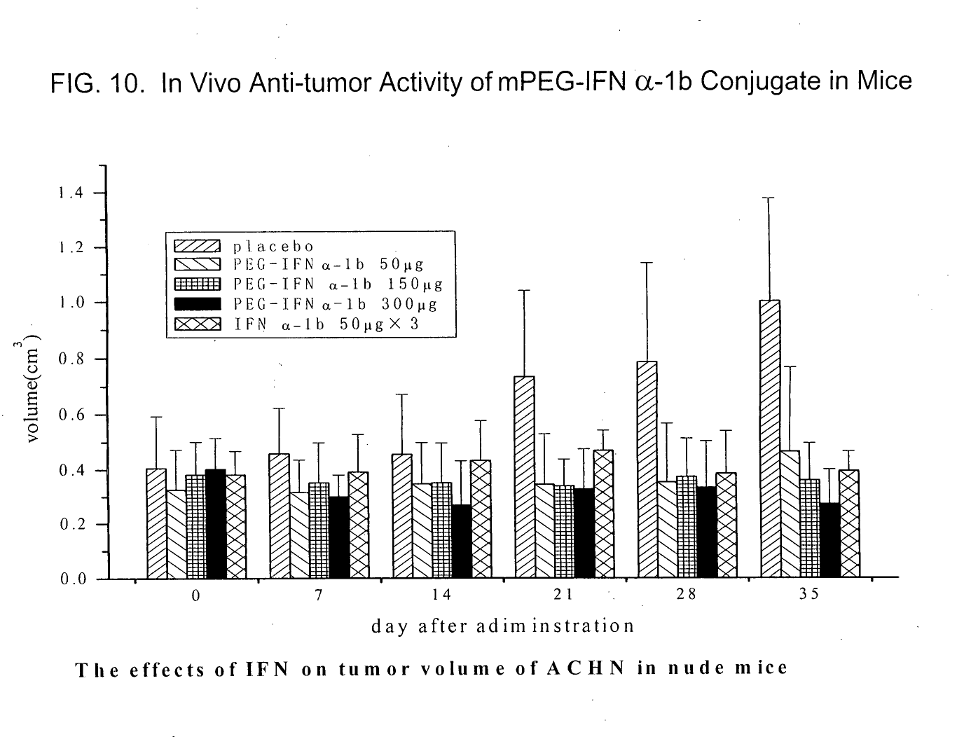

As shown in FIG. 10, in the first four weeks of the treatment, mPEG (20 kD)-IFN α-1 b and IFN α-1b significantly inhibited the tumor growth of the mice implanted with ACHN tumor cells, as compared with the placebo control group. In the fifth week of the treatment, an initial dose response of mPEG (20 kD)-IFN α-1b on the inhibition of tumor growth was observed. The inhibitions of tumor growth were similar between once weekly injection of 150 μg of mPEG (20 kD)-IFN α-1b and thrice weekly injection of 50 μg of IFN α-1b.

Having now fully described this invention, it will be appreciated that by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional method steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

What is claimed is:

1. A polyol-interferon-α conjugate having a polyol moiety covalently bound to Cys⁸⁶ of human interferon α-1b.

2. The polyol-interferon-α conjugate according to claim 1, wherein the interferon α-1b is isolated from human cells or tissues.

3. The polyol-interferon-α conjugate according to claim 1, wherein the interferon α-1b is a recombinant protein.

4. The polyol-interferon-α conjugate according to claim 3, wherein the interferon α-1b is expressed in a host selected from the group consisting of a bacterial cell, a fungal cell, a plant cell, an animal cell and an insect cell, a yeast cell, and a transgenic animal.

5. The polyol-interferon-α conjugate according to claim 2 or claim 3, wherein the interferon α-1b comprises the amino acid sequence set forth in SEQ ID. NO. 2.

6. The polyol-interferon-α conjugate according to claim 2 or claim 3, wherein the interferon α-1b comprises a homologue, ortholog, variant, analog, derivative, biologically active fragment, pharmaceutically active fragment or mutation of the amino acid sequence set forth in SEQ ID. NO. 2.

7. The polyol-interferon-α conjugate according to claim 2 or claim 3, wherein the interferon α-1b is encoded by a polynucleotide having the DNA sequence set forth in SEQ I.D. No. 1.

8. The polyol-interferon-α conjugate according to claim 1, wherein the polyol moiety is a polyethylene glycol moiety.

9. The polyol-interferon-α conjugate according to claim 1, wherein the polyol moiety is a single chain polyol moiety.

10. The polyol-interferon-α conjugate according to claim 1, wherein the polyol moiety is a branched chain polyol moiety.

11. The polyol-interferon-α conjugate according to claim 1, wherein the polyol moiety is a polyalkylene glycol moiety.

12. The polyol-interferon-α conjugate according to claim 1, wherein the polyol-interferon α-1b conjugate has the same or higher in vivo interferon-α activity as native human interferon α-1b.

13. The polyol-interferon-α conjugate according to claim 1, wherein the polyol-interferon α-1b conjugate has a homogenous molecular weight.

14. A pharmaceutical composition comprising a polyol-interferon-α conjugate having a polyol moiety covalently bound to Cys⁸⁶ of human interferon α-1b and a pharmaceutically acceptable carrier, excipient or auxiliary agent.

15. The pharmaceutical composition according to claim 14, wherein the polyol moiety is a polyethylene glycol moiety.

16. The pharmaceutical composition according to claim 14, wherein the polyol moiety is a single chain polyol moiety.

17. The pharmaceutical composition according to claim 14, wherein the polyol moiety is a branched chain polyol moiety.

18. The pharmaceutical composition according to claim 14, wherein the polyol moiety is a polyalkylene glycol moiety.

19. A method for producing a polyol-interferon conjugate comprising the steps of:
providing an interferon, wherein said interferon comprises a single free cysteine;
providing a maleimide polyol; and
contacting the interferon with the maleimide polyol, wherein the maleimide polyol forms a covalent thioether bond with the free cysteine, thereby producing a polyol-interferon conjugate.

20. The method of claim 19, wherein the interferon is a human alpha interferon.

21. The method of claim 20, wherein the alpha interferon is recombinant human interferon α-1b.

22. The method of claim 21, wherein the interferon α-1 b comprises the amino acid sequence set forth in SEQ ID. NO. 2.

23. The method of claim 21, wherein the interferon α-1b comprises a homologue, ortholog, variant, analog, derivative, biologically active fragment, pharmaceutically active fragment or mutation of the amino acid sequence set forth in SEQ ID. NO. 2.

24. The method of claim 19, wherein the interferon is selected from the group consisting of:
a naturally occurring interferon, a genetically engineered interferon and a chimeric interferon.

25. The method of claim 19, wherein a cysteine residue comprises the single free thiol group.

26. The method of claim 25, wherein the interferon further comprises disulfide bonded cysteine residues.

27. The method of claim 21, wherein Cys⁸⁶ of human interferon α-1b comprises the single free thiol group.

28. A method of modulating a process mediated by interferon-α comprising administering to a patient an effective amount of a polyol-interferon-α conjugate according to claim 1.

29. The method of claim 28, wherein the process mediated by interferon-α comprises inflammation, viral infection, bacterial infection or cancer.

30. A method of treating a patient with an interferon-α-responsive condition or disease, comprising administering to a patient an effective amount of the polyol-interferon-α conjugate of claim 1 or the polyol-interferon conjugate prepared according to the method of claim 19.

31. The method of claim 30, wherein the patient suffers from an inflammatory disorder, a viral infection, a bacterial infection or cancer.

32. The method of claim 29 or claim 31, wherein the viral infection comprises hepatitis C infection, hepatitis B infection or HIV infection.

33. The method of claim 31, wherein the inflammatory disorder is multiple sclerosis, arthritis, asthma, cystic fibrosis, or interstitial lung disease.

34. The method of claim 29 or clam 31, wherein the cancer is selected from myeloma, lymphoma, liver cancer, breast cancer, melanoma, and hairy-cell leukemia.

35. A method for purifying a polyol-interferon α-1b conjugate comprising:
(a) contacting a polyol-interferon α-1b conjugate with a hydrophobic interaction chromatography resin wherein the polyol-interferon α-1b conjugate binds to the chromatography resin;
(b) eluting the polyol-interferon α-1b conjugate from the hydrophobic interaction chromatography resin;
(c) applying the eluted polyol-interferon α-1b conjugate to a size exclusion chromatography column; and
(d) collecting purified polyol-interferon α-1b conjugate from the size exclusion chromatography column, thereby purifying the polyol-interferon α-1b.

36. The method of claim 35, further comprising concentrating the eluted polyol-interferon α-1b conjugate of step (b) prior to applying to the size exclusion chromatography column of step (c).

37. The method of claim 36, wherein said concentrating comprises ultrafiltration and diafiltration.

38. The method of claim 35, wherein the hydrophobic interaction chromatography resin is a butyl agarose resin.

39. The method of claim 35, wherein the size exclusion chromatography column comprises cross-linked agarose, dextran or a mixture thereof.


SEQUENCE LISTING (2 sequences)

SEQ ID NO: 1 - DNA, 504 bp, Homo sapiens
atgtgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 2 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE